(19) United States
(12) Patent Application Publication
(10) Pub. No.: US 2023/0076415 A1
(43) Pub. Date: Mar. 9, 2023

(54) METHODS AND COMPOSITIONS FOR TREATING MELANOMA

(71) Applicants: INSERM (INSTITUT NATIONAL DE LA SANTÉ ET DE LA RECHERCHE MÉDICALE), Paris (FR); UNIVERSITE COTE D'AZUR, Nice (FR)

(72) Inventors: Marcel DECKERT, NICE (FR); Mickael OHANNA, Nice (FR); Pierric BIBER, Nice (FR); Sophie TARTARE-DECKERT, Nice (FR)

(21) Appl. No.: 17/793,159

(22) PCT Filed: Jan. 15, 2021
(86) PCT No.: PCT/EP2021/050815

§ 371 Date: Jul. 15, 2022

(30) Foreign Application Priority Data
Jan. 17, 2020 (EP) 20305037.2

(51) Int. Cl. C12Q 1/6886; A61K 31/47; A61K 31/7052; A61P 35/00

(52) U.S. Cl. CPC C12Q 1/6886; A61K 31/47; A61K 31/7052; A61P 35/00; C12Q 2600/178

(57) ABSTRACT

Accordingly, the invention relates to a method for predicting the survival time of a subject suffering from melanoma by quantifying the expression level of PSMD14 in a biological sample.

### FIELD OF THE INVENTION

The invention is in the field of oncology, more particularly the invention relates to a method and compositions for treating melanoma.

### BACKGROUND OF THE INVENTION

Cutaneous melanoma is the deadliest form of skin cancer whose incidence is rapidly increasing worldwide. Melanoma develops from the malignant transformation of melanocytes (1,2). Unless surgically removed, melanoma rapidly progresses to a very aggressive disease characterized by a metastatic potential and poor therapeutic response. The majority of human melanomas display genetic alterations in BRAF and NRAS genes, leading to constitutive activation of MEK/ERK mitogen-activated protein kinase (MAPK) pathway (3). In addition, melanomas are characterized by a high level of differentiation state plasticity and intratumor heterogeneity (4,5), which are sources of drug-tolerant cells and relapse (5,6). Other (epi)genetic changes and signals from the tumor microenvironment affect the survival, proliferation and invasive potential of melanoma cells by altering additional signaling pathways (7,8).

BRAF inhibitors (BRAFi), MEK inhibitors (MEKi), or their combination, which are prescribed for the treatment of patients with BRAFV600E/K mutant melanoma, elicit high response rates. Nevertheless, drug resistance invariably develops within 6 to 12 months of treatment (9,10). Secondary mutations affecting components of the MAPK pathway, the activation of parallel signaling pathways such as the PI3K/AKT pathway, tumor heterogeneity and microenvironmental cues are associated with acquired resistance and relapses (7,11-14). In cases of melanoma with no BRAF mutations (more than 50% of the total), treatment with antibodies blocking the functions of the immune checkpoints PD-1 and CTLA-4 has become a first line therapeutic regimen (15). Immunotherapy also appears as a second line of treatment in the cases of innate and acquired resistance to BRAF/MEK inhibitors. However, most patients do not fully respond to immunotherapies. Globally, the majority of patients with metastatic melanoma will need additional treatments (15). Therefore, to further clarify the mechanism underlying melanoma pathogenesis is of great importance for improving the efficacy of melanoma therapy and the management of melanoma patients.

The ubiquitin-proteasome system (UPS) plays a critical role in protein quality control and cancer proteostasis (16,17). The UPS is composed of proteolytic multiprotein complexes called proteasomes acting downstream a cascade of E1, E2 and E3 enzymes that activate ubiquitin residues and add them to mutant/aged/misfolded proteins targeted for destruction by proteasomes (17). The removal of ubiquitin moieties from protein substrates is catalyzed by a family of more than 90 ubiquitin-specific proteases called deubiquitinases (DUBs) that participate to key cellular processes (18,19). Evidences suggest that aberrant expression, mutations or dysfunction of DUBs are associated with the pathogenesis of neoplastic diseases (20), underscoring the therapeutic potential of targeting DUBs in cancer. In melanoma, the mutation of genes associated with ubiquitination, such as BAP1, FBXW7 and PARK2, are frequently identified (21). Several DUBs have been directly implicated in melanoma tumorigenesis and therapeutic response. For example, USP13 was shown to regulate the stability of the microphthalmia-associated transcription factor (MITF) that is essential for melanocyte development and melanogenesis (22). In NRAS mutant melanomas, USP9X has been identified as an important modulator of NRAS expression via the stabilization of ETS-1 (23). In BRAF mutant melanoma cells, USPS inhibition promoted p53 and FAS expression, thereby potentiating targeted therapies (24). Other DUBs such as USP8 (25) and USP15 (26) are overexpressed in melanoma and participate to disease progression or antitumor responses. Some DUBs are intrinsic components of the proteasome (35). In this context, we have shown that targeting USP14 exerts a broad anti-melanoma effect via a strong reticulum stress and a caspase-independent cell death (27). In the other hand, the repression of CPLD by the EMT transcription factor Snail1 resulted in proliferation and invasion of melanoma cells (28), further illustrating the complexity of DUB functions in melanoma pathogenesis. To date, the role of DUBs in melanoma remains undefines in to the control of survival/proliferation processes.

### SUMMARY OF THE INVENTION

The invention relates to a method for treating melanoma in a subject in need thereof comprising a step of administering said subject with a therapeutically effective amount of an inhibitor of PSMD14. In particular, the present invention is defined by the claims.

### DETAILED DESCRIPTION OF THE INVENTION

Inventors have used an unbiaised genetic screen to identify novel DUBs involved in melanoma cell proliferation and/or survival. A human siRNA library targeting 98 known DUBs was transfected into 501Mel melanoma cell cultures and real-time analysis of cell proliferation was performed. This screen led us to identify PSMD14 (POH1/RPN11) as a novel key regulator of melanoma cell viability and drug resistance. Inventors found that elevated expression of PSMD14 is associated with metastatic melanoma (MM) and a poorer prognosis, indicating that PSMD14 can be used as a biomarker. They further shown that siRNA-mediated knockdown and pharmacological inhibition of PSMD14 impair in vitro proliferation and viability of melanoma cells harbouring different mutational status, and reduced the growth of BRAF and NRAS mutant mouse melanomas in vivo. They also provided evidence that PSMD14 is a critical regulator of melanoma early adaptation to BRAFi/MEKi and shown that the PSMD14 inhibitor quinolone-8-thiol (8-TQ) overcomes acquired resistance to MAPK-targeting therapies in vitro and in vivo. They demonstrated that contrary to another proteasome-associated DUB, USP14, which regulates melanoma viability via a p53-independent ER stress response and protein degradation (27), targeting PSMD14 in melanoma exerts its cytotoxic action via a p53-dependent DNA damage response and a non-proteolytic modulation of H2A histones activity. Importantly, the two proteasome-associated enzymes, USP14 and PSMD14 belong to two different classes of deubiquitinases (DUBs) with different catalytic activities: while USP14 is an ubiquitin-specific isopeptidase, PSMD14 is an ubiquitin-specific zinc metalloprotease. Finally, the inventors provided evidence that targeting PSMD14 in vitro has a synergistic action with the combination of BRAFi and MEKi to dramatically impair BRAF mutant melanoma cell viability. Together, their findings indicate that PSMD14 is a promising therapeutic target in melanoma.

### Method for Predicting the Survival Time of a Subject Suffering from Melanoma

In a first aspect, the invention relates to a method for predicting the survival time of a subject suffering from melanoma comprising the steps of i) quantifying the expression level of PSMD14 in a biological sample obtained from the subject; ii) comparing the expression level quantified at step i) with its predetermined reference value and iii) concluding that the subject will have a short survival time when the expression level of PSMD14 is higher than its predetermined reference value or concluding that the subject will have a long survival time when the expression level of PSMD14 is lower than its predetermined reference value.

In a particular embodiment, the invention relates to a method for predicting the survival time of a subject suffering from resistant melanoma comprising the steps of i) quantifying the expression level of PSMD14 in a biological sample obtained from the subject; ii) comparing the expression level quantified at step i) with its predetermined reference value and iii) concluding that the subject will have a short survival time when the expression level of PSMD14 is higher than its predetermined reference value or concluding that the subject will have a long survival time when the expression level of PSMD14 is lower than its predetermined reference value.

The method is particularly suitable for predicting the duration of the overall survival (OS), progression-free survival (PFS) and/or the disease-free survival (DFS) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they have become cancer-free (achieved remission). DFS gives more specific information and is the number of people with a particular cancer who achieve remission. Also, progression-free survival (PFS) rates (the number of people who still have cancer, but their disease does not progress) include people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye. In a particular embodiment, the melanoma is cutaneous melanoma.

As used herein, the term “resistant melanoma” also called as metastatic melanoma refers to melanoma which does not respond to a treatment. The cancer may be resistant at the beginning of treatment or it may become resistant during treatment. The resistance to drug leads to rapid progression of metastatic of melanoma. The resistance of cancer for the medication is caused by mutations in the gene which are involved in the proliferation, divisions or differentiation of cells. In the context of the invention, the resistance of melanoma is caused by the mutations (single or double) in the following genes: BRAF, MEK or NRAS. The resistance can be also caused by a double-negative BRAF and NRAS mutation.

As used herein, the term “subject” denotes a mammal, such as a rodent, a feline, a canine, and a primate. Particularly, the subject according to the invention is a human. More particularly, the subject according to the invention has or is susceptible to have melanoma. In particular embodiment, the subject has or is susceptible to have cutaneous melanoma. In a particular embodiment, the subject has or is susceptible to have resistant and/or metastatic melanoma.

As used herein, the term “PSMD14” also known as 26S proteasome non-ATPase regulatory subunit 14 or 26S proteasome non-ATPase subunit is a Zn2+-dependent metalloisopeptidase that is associated to the 19S regulatory particle of the proteasome. PSMD14 is an enzyme that in humans is encoded by the PSMD14 gene. The naturally occurring human PSMD14 gene has a nucleotide sequence as shown in Genbank Accession number NM_005805 and the naturally occurring human PSMD14 protein has an amino acid sequence as shown in Genbank Accession number NP 005796. The murine nucleotide and amino acid sequences have also been described (Genbank Accession numbers NM_021526 and NP 067501). PSMD14 is a component of a multiprotein complex that catalyzes the degradation of ubiquitinated intracellular proteins, thus having a key role in the maintenance of protein homeostasis by removing misfolded or damaged proteins, which could impair cellular functions, and by removing proteins whose functions are no longer required.

As used herein, the term “expression level” refers to the expression level of PSMD14. Typically, the expression level of the PSMD14 gene may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50 formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

As used herein, the term “biological sample” refers to any sample obtained from a subject, such as a serum sample, a plasma sample, a urine sample, a blood sample, a lymph sample, or a tissue biopsy. In a particular embodiment, biological sample for the determination of an expression level include samples such as a blood sample, a lymph sample, or a biopsy.

In a particular embodiment, the biological sample is a blood sample, more particularly, peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

In a particular embodiment, the biological sample is a tumor tissue sample. More particularly, in a particular embodiment, the biological sample is normal, primary or melanoma tissue.

As used herein, the term “tumor tissue sample” has its general meaning in the art and encompasses pieces or slices of tissue that have been removed including following a surgical tumor resection. The tumor tissue sample can be subjected to a variety of well-known post-collection preparative and storage techniques (e.g., fixation, storage, freezing, etc.) prior to determining the cell densities. Typically the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides). The tumour tissue sample can be used in microarrays, called as tissue microarrays (TMAs). TMA consists of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

In some embodiments, the expression level of PSMD14 is determined by Immunohistochemistry (IHC).

Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex.

Accordingly, the tumor tissue sample is firstly incubated with the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the maker of interest. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker, ii) proceeding to digitalisation of the slides of step i). by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity or the absolute number of stained cells in each unit whereby the number or the density of cells stained of each unit is assessed.

As used herein, the term “the predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER. SAS, DESIGNROC.FOR, MULTIREADER POWER. SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of

a) providing a collection of tumor tissue samples from subject suffering from melanoma;

b) providing, for each tumor tissue sample provided at step a), information relating to the actual clinical outcome for the corresponding subject (i.e. the duration of the disease-free survival (DFS) and/or the overall survival (OS));

c) providing a serial of arbitrary quantification values;

d) quantifying the cell density for each tumor tissue sample contained in the collection provided at step a);

e) classifying said tumor tissue samples in two groups for one specific arbitrary quantification value provided at step c), respectively: (i) a first group comprising tumor tissue samples that exhibit a quantification value for level that is lower than the said arbitrary quantification value contained in the said serial of quantification values; (ii) a second group comprising tumor tissue samples that exhibit a quantification value for said level that is higher than the said arbitrary quantification value contained in the said serial of quantification values; whereby two groups of tumor tissue samples are obtained for the said specific quantification value, wherein the tumor tissue samples of each group are separately enumerated;

f) calculating the statistical significance between (i) the quantification value obtained at step e) and (ii) the actual clinical outcome of the subjects from which tumor tissue samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 tumor tissue samples of 100 subjects. The 100 samples are ranked according to the cell density. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

### Method for Treating Melanoma

Inventors have shown that an inhibition of PSMD14 by siRNAs and pharmacological inhibitor, the cell proliferation of melanoma cell drastically decreased.

Accordingly, in a second aspect, the invention relates to a method for treating melanoma in a subject in need thereof comprising a step of administering said subject with a therapeutically effective amount of an inhibitor of PSMD14.

In a particular embodiment, the subject is identified as having a short survival time by performing the method as described above.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The term “inhibitor of PSMD14” refers to a natural or synthetic compound that has a biological effect to inhibit the activity or the expression of PSMD14. More particularly, such compound by inhibiting PSMD14 activity induces a rapid accumulation of K48-linked poly-ubiquitinated proteins, the down-regulation of the cell-cycle dependent kinase CDK2, a strong induction of the CDK inhibitor p21Cip1, of p53 expression and of H2AX serine 139 phosphorylation. In a particular embodiment, the inhibition of PSMD14 leads to a DNA damage response in melanoma associated with DNA double strand breaks and increased expression of the apoptosis-associated markers cleaved PARP and cleaved caspase-3. In a further embodiment, inhibition of PSMD14 expression and/or activity induces a suppression of melanoma cell proliferation and viability of melanoma cell.

In a particular embodiment, the inhibitor of PSMD14 is a peptide, peptidomimetic, small organic molecule, antibody, aptamers, siRNA or antisense oligonucleotide. The term “peptidomimetic” refers to a small protein-like chain designed to mimic a peptide. In a particular embodiment, the inhibitor of PSMD14 is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of PSMD14 is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the small molecule is quinoline-8-thiol (8TQ) also called as 8-Mercaptoquinoline and its derivatives. This small organic molecule has the formula C9H7NS, CAS number: 34006-16-1 and the following structure in the art:

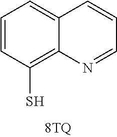 [SMILES: Sc1cccc2cccnc12]

In a particular embodiment, the small molecule is derivates of capzimin as described in Li J et al 2017, Nat Chem Biol. 2017 May; 13(5):486-493.

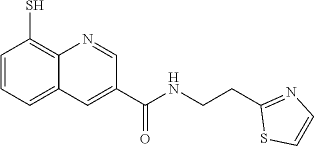 [SMILES: O=C(NCCc1nccs1)c1cnc2c(S)cccc2c1]

In a particular embodiment, the small molecule is derivates of epidithiodiketopiperazine as described in Li J et al 2018, Cell Chem Biol. 2018 Nov. 15 (25):1350-1358.

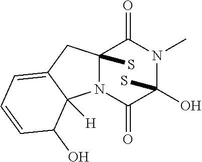

In some embodiments, the inhibitor of PSMD14 expression is a short hairpin RNA (shRNA), a small interfering RNA (siRNA) or an antisense oligonucleotide which inhibits the expression of PSMD14. In a particular embodiment, the inhibitor of PSMD14 expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. In a particular embodiment, the inhibitor of PSMD14 expression is an anti-sense oligonucleotides (ASO). Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PSMD14 expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the inhibitor of PSMD14 is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the inhibitor is an intrabody having specificity for PSMD14. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

### Method for Treating Resistant Melanoma

Acquired resistance to targeted therapies is currently a clinical challenge in the treatment of advanced metastatic melanoma. Therefore, inventors examined the impact of targeting PSMD14 in melanoma cells resistant to BRAFV600E inhibitors (BRAFi). They have shown that melanoma treatment with pharmacological inhibitors against PSMD14 can overcome resistance to drugs targeting oncogenic BRAF.

Accordingly, in a third aspect, the invention relates to a method for treating resistant melanoma in a subject in need thereof comprising a step of administering said subject with a therapeutically effective amount of an inhibitor of PSMD14.

As used herein, the term “resistant melanoma” refers to melanoma which does not respond to a treatment. The cancer may be resistant at the beginning of treatment or it may become resistant during treatment. The resistance to drug leads to rapid progression of metastatic of melanoma. The resistance of cancer for the medication is caused by mutations in the gene which are involved in the proliferation, divisions or differentiation of cells. In the context of the invention, the resistance of melanoma is caused by the mutations (single or double) in the following genes: BRAF, MEK or NRAS. The resistance can be also caused by a double-negative BRAF and NRAS mutation.

In a particular embodiment, the melanoma is resistant to a treatment with the inhibitors of BRAF mutations. BRAF is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T5981, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E.

The inhibitors of BRAF mutations are well known in the art. In a particular embodiment, the melanoma is resistant to a treatment with vemurafenib. Vemurafenib also known as PLX4032, RG7204 ou RO5185426 and commercialized by Roche as Zelboraf. In a particular embodiment, the melanoma is resistant to a treatment with dacarbazine. Dacarbazine also known as imidazole carboxamide is commercialized as DTIC-Dome by Bayer. In a particular embodiment, the melanoma is resistant to a treatment with dabrafenib also known as tafinlar which is commercialized by Novartis.

In a further embodiment, the melanoma is resistant to a treatment with the inhibitors of MEK. MEK refers to Mitogen-activated protein kinase kinase, also known as MAP2K, MEK, MAPKK. It is a kinase enzyme which phosphorylates mitogen-activated protein kinase (MAPK). MEK is activated in melanoma. The inhibitors of MEK are well known in the art. In a particular embodiment, the melanoma is resistant to a treatment with trametinib also known as mekinist which is commercialized by GSK. In a particular embodiment, the melanoma is resistant to a treatment with cobimetinib also known as cotellic commercialized by Genentech. In a particular embodiment, the melanoma is resistant to a treatment with Binimetinib also knowns as MEK162, ARRY-162 is developed by Array Biopharma.

In a particular embodiment, the melanoma is resistant to a treatment with the inhibitors of NRAS. The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations. In a particular embodiment, the melanoma is resistant in which double-negative BRAF and NRAS mutant melanoma.

In a particular embodiment, the melanoma is resistant to a combined treatment. As used herein, the terms “combined treatment”, “combined therapy” or “therapy combination” refer to a treatment that uses more than one medication. The combined therapy may be dual therapy or bi-therapy. In the context of the invention, the melanoma is resistant to a combined treatment characterized by using an inhibitor of BRAF mutation and an inhibitor of MEK as described above. For example, the combined treatment may be a combination of vemurafenib and cotellic.

In a further embodiment, the melanoma is resistant to a treatment with an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-napthoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-napthoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-éthyl]amino}-1,2,5-oxadiazole-3 carboximidamide:

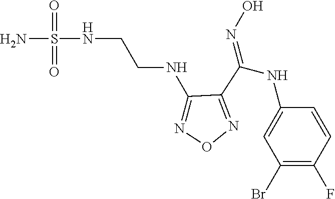 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7 S)-6,7,8,9-tetrahydro-7-(1-pyrrolidinyl)-5H-benzocyclohepten-2-yl]- and has the following formula in the art:

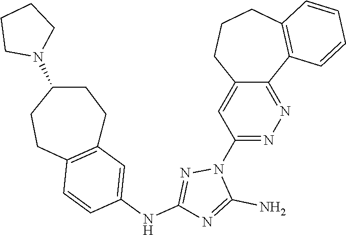 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “subject” denotes a mammal, such as a rodent, a feline, a canine, and a primate. Particularly, the subject according to the invention is a human. More particularly, the subject according to the invention has or susceptible to have melanoma. In a particular embodiment, the subject has or susceptible to have melanoma resistant to at least one of the treatments as described above. The subject having a melanoma resistant is identified by standard criteria. The standard criteria for resistance, for example, are Response Evaluation Criteria In Solid Tumors (RECIST) criteria, published by an international consortium including NCI.

As used herein the terms “administering” or “administration” refer to the act of injecting or otherwise physically delivering a substance as it exists outside the body (e.g., an inhibitor of PSMD14) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of inhibitor of PSMD14 for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the melanoma is resistant to a treatment with a combination of MEK inhibitors and/or immune checkpoint inhibitor as described above.

### Combined Preparation

In a fourth aspect, the invention relates to i) an inhibitor of PSMD14 and ii) a classical treatment used as a combined preparation for use in the prevention and/or treatment of melanoma and/or resistant melanoma in a subject in need thereof.

In a particular embodiment, the invention relates to i) an inhibitor of PSMD14 and ii) a classical treatment for use by simultaneous, separate or sequential administration in the prevention and/or treatment melanoma and/or resistant melanoma in a subject in need thereof.

As used herein, the terms “combined treatment”, “combined therapy” or “therapy combination” refer to a treatment that uses more than one medication. The combined therapy may be a tri-therapy combining a PSMD14 inhibitor, a BRAF inhibitor and a MEK inhibitor. In a particular embodiment, the PSMD14 inhibitor is 8TQ, the BRAF inhibitor is vemurafenib and MEK inhibitor is cobimetinib.

As used herein, the term “administration simultaneously” refers to administration of at least 2 or 3 active ingredients by the same route and at the same time or at substantially the same time. The term “administration separately” refers to an administration of at least 2 or 3 active ingredients at the same time or at substantially the same time by different routes. The term “administration sequentially” refers to an administration of at least 2 or 3 active ingredients at different times, the administration route being identical or different.

As used herein, the term “classical treatment” refers to treatments well known in the art and used to treat melanoma. In the context of the invention, the classical treatment refers to targeted therapy, radiation therapy, immunotherapy or chemotherapy.

In a particular embodiment, the invention relates to i) an inhibitor of PSMD14 according to the invention and ii) a targeted therapy used as a combined preparation for use in the prevention and/or treatment of melanoma and/or resistant melanoma in a subject in need thereof.

In a particular embodiment, the invention relates to i) an inhibitor of PSMD14 ii) an inhibitor of BRAF and iii) an inhibitor of MEK as a combined preparation for use in the prevention and/or treatment of melanoma and/or resistant melanoma in a subject in need thereof.

In a particular embodiment, the invention relates to i) an inhibitor of PSMD14 ii) an inhibitor of BRAF and iii) an inhibitor of MEK for use by simultaneous, separate or sequential administration in the prevention and/or treatment melanoma and/or resistant melanoma in a subject in need thereof.

As used herein, the term “targeted therapy” refers to drugs which attack specific genetic mutations within cancer cells, such as melanoma while minimising harm to healthy cells. Typically, the targeted therapy for melanoma refers to use of BRAF, MEK or NRAS inhibitors as described above.

In a particular embodiment, the invention relates to i) an inhibitor of PSMD14 according to the invention and ii) a radiation therapy used as a combined preparation for use in the prevention and/or treatment of melanoma and/or resistant melanoma in a subject in need thereof.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates to i) an inhibitor of PSMD14 according to the invention and ii) a chemotherapy used as a combined preparation for use in the prevention and/or treatment of melanoma and/or resistant melanoma in a subject in need thereof.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalamycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defo famine; demecolcine; diaziquone; eflornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylamine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the invention relates to i) an inhibitor of according to the invention and ii) an immune checkpoint inhibitor as described above, used as a combined preparation for use in the prevention and/or treatment of a melanoma and/or resistant melanoma in a subject in need thereof.

### Pharmaceutical Composition

The inhibitors of PSMD14 as described above may be combined with pharmaceutically acceptable excipients, and optionally sustained-release matrices, such as biodegradable polymers, to form pharmaceutical compositions.

Accordingly, in a fifth aspect, the invention relates to a pharmaceutical composition comprising inhibitors of PSMD14 and pharmaceutically acceptable excipients. In some embodiments, the invention relates to pharmaceutical composition comprising an inhibitor of PSMD14, an inhibitor of BRAF, an inhibitor of MEK and a pharmaceutically acceptable excipient. In a particular embodiment, the inhibitor of PSMD14 is 8TQ, the inhibitor of BRAF is vemurafenib and the inhibitor of MEK is cobimetinib.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Method of Screening

A further object of the present invention relates to a method of screening a drug suitable for the treatment of melanoma comprising i) providing a test compound and ii) determining the ability of said test compound to inhibit the activity of PSMD14.

Any biological assay well known in the art could be suitable for determining the ability of the test compound to inhibit the activity of PSMD14. In some embodiments, the assay first comprises determining the ability of the test compound to bind to PSMD14. In some embodiments, a population of cells is then contacted and activated so as to determine the ability of the test compound to inhibit the activity of PSMD14. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PSMD14, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1. Identification of PSMD14 as a novel target and prognostic factor in melanoma. Kaplan-Meier overall survival curves in melanoma patients with high or low PSMD14 expression. Data from TGCA (Skin Cutaneous melanoma (SKCM) dataset) were obtained through SurvExpress (p=0.0079; log-rank test).

FIG. 2. PSMD14 depletion has a potent cytotoxic effect on melanoma cells. A) Experimental validation of the siDUB screening on melanoma cell proliferation. 501Mel and A375 melanoma cells were plated 10,000 cells/well in a 24-well plate and transfected with a control siRNA (siCTRL) or 3 different siRNA sequences targeting PSMD14 (siPSMD14 #1, #2 and #3). Cell confluence was imaged for 48 h using the IncuCyte Zoom live imaging system. Curves represent normalized cell proliferation of each siRNA relative to siCTRL. Data are the mean±SEM (n=3). ***P<0.001, 2-way ANOVA analysis. B) Colony survival analysis after PSMD14 depletion (siPSMD14 #1, #2 and #3) compared to control siRNA (siCTRL). 501Mel and A375 cells were transfected with siRNAs, re-seeded at 2,000 cells/well and grown for 1 week. Colonies were stained with crystal violet. Histograms showed the quantification of crystal violet absorbance at 595 nm. Data are the mean of three independent experiments performed in duplicate (±SEM). ***P<0.001. C) Survival analysis performed on a subset of melanoma cells after PSMD14 depletion. Cells from primary and metastatic melanomas were transfected with siCTRL or siPSMD14 #1. After 72h, cells were stained with crystal violet. Box plots show the mean±SEM of crystal violet quantification relative to siCTRL (n=3, ***P<0.001.). D) Effect of PSMD14 depletion on melanoma cell viability. A375 cells were transfected with siCTRL or siPSMD14 #1. After 72h, cells were labeled with Annexin V-FITC and DAPI and analysed by flow cytometry. Histograms show the percentage of cell death (Annexin V and DAPI positive cells) (mean±SEM, n=3).

FIG. 3. Pharmacological inhibition of PSMD14 impairs melanoma cell survival. A) Survival analysis performed on A375 cells treated with vehicle or increasing doses of 8TQ (0.27, 0.9, 3 μM). After 72h, cells were stained with crystal violet. Right, cell viability was quantified on lysates by analyzing crystal violet staining at 595 nm. Histograms represent the mean±SD of three independent experiments in duplicate (***P<0.001; ns, non significant). B) Effect of PSMD14 inhibition on melanoma cell viability. A375 cells were treated with vehicle or increasing doses of 8TQ (0.27, 0.9, 3 μM). After 72h, cells were stained with Annexin V-FITC and DAPI and analysed by flow cytometry. Histograms show the percentage of cell death (Annexin V and DAPI positive cells) (mean±SEM, n=3). The calculated mean IC50±sem is shown. (** P<0.005, ***P<0.001). C) A subset of melanoma cells was treated with increasing doses of 8TQ. After 72h, cells were labeled and analyzed as in b). The corresponding IC50 of 8TQ was determined by measuring cell viability. Box plots indicating the upper/lower quartile and the median with whiskers from min to max values from 2 experiments performed in triplicate.

FIG. 4. The PSMD14 inhibitor 8TQ inhibits tumor growth in melanoma syngeneic models. A) 2×105 murine melanoma cells Yumm1.7 (derived from BrafV600E/Pten null/cdkn2a null mice) were subcutaneously inoculated into both flanks of 6-week old C57Bl/6J female mice. After one week, mice were injected i.p 3 times per week with 8TQ (10 mg/kg) or vehicle. The tumor size was measured with a caliper and the tumor volume was determined. Data shown are mean±SD of tumor volume (n=12; ***, ***P<0.001, 2way ANOVA). B) 4×105 murine melanoma MaNRas (NRasQ61K) were subcutaneously injected into both flanks of 6-week old C57Bl/6J female mice. After two weeks, mice were injected i.p every two days with 8TQ (10 mg/kg) or vehicle. The tumor size was measured with a caliper and the tumor volume was determined. Data shown are mean±sd of tumor volume (n=12; ***, ***P<0.001, 2way ANOVA).

FIG. 5. Targeting PSMD14 overcomes resistance to MAPK pathway inhibition. A) Melanoma cell lines resistant to BRAFi (A375RIV, M229R, M238R and M249R) or double resistant to BRAFi and MEKi (A375DDR) were transfected with siCTRL or siPSMD14 #1. After 72h, cell colonies were stained with crystal violet and the absorbance at 595 nm was measured. Box plots show the upper/lower quartile and the median with whiskers from min to max values (n=3, ***P<0.001). B) The above panel of melanoma cells was treated with increasing doses of 8TQ. After 72h, cells were labeled and analyzed. The corresponding IC50 of 8TQ was determined. Box plots indicate the upper/lower quartile and the median with whiskers from min to max values from 2 experiments performed in triplicate. C) 1×106 A375Riv human melanoma cells were implanted into both flanks of nude mice. After 2 days, mice were injected i.p every day with 8TQ (10 mg/kg) or vehicle. The tumor size was measured with a caliper and the tumor volume was determined. Data shown are mean±SD of tumor volume (n=12; ***, ***P<0.001, 2way ANOVA).

FIG. 6. PSMD14 inhibition triggers a p53-Dependent DNA damage response in melanoma. A) A375 cells were transfected with control siRNA (siCTRL) or siRNA targeting PSMD14 for 72h and stained for phosphorylated H2AX (pH2AX). Bar graphs show the quantification of pH2AX expression by immunofluorescence analysis. At least 200 cells were counted from each condition. Data are the mean±SD (n=3). B) Survival analysis in A375 cells transfected for 72h with siCTRL, siPSMD14, sip53 or the combination of siPSMD14 and sip53. Left, representative images of crystal violet stainings are shown. Right, cell viability was quantified on lysates by analyzing crystal violet staining at 595 nm. Histograms represent the mean±SD of three independent experiments in duplicate (***P<0.001). C) Survival analysis in A375 cells transfected for 48h with siCTRL or siPSMD14 in the presence or not of 100 μM H2O2. Cell viability was quantified as above. Histograms represent the mean±SD of three independent experiments in duplicate (***P<0.001).

FIG. 7. Targeting PSMD14 has a synergistic action with the combination of BRAFi and MEKi on BRAF mutant melanoma cell viability. A375 cells were transfected with control siRNA (siCTRL) or siRNA targeting PSMD14, and cultivated in the presence or not of a combination of BRAFi (Vemurafenib, 1 μM) and MEKi (Cobimetinib, 0.1 μM). Cell death was imaged for 24h using the Red Cytotox fluorescent reagent and the IncuCyte ZOOM™ live imaging system. Relative cell death was then quantified with the IncuCyte integrated analysis software. Curves represent normalized cell death signal of each condition relative to siCTRL.

### EXAMPLE

Materials and Methods

Cell Culture

MeWo and WM793 melanoma cell lines were from ATCC. WM164 cells were purchased from Rockland. WM9 and Sbcl2 cells were provided by M. Herlyn. M229, M238 and M249 cells and their BRAF inhibitor-resistant sublines were from R. Lo and were previously characterized (11). 501Mel cells were a gift from R. Halaban. A375 and their BRAF inhibitor-resistant derivative (A375Riv) were provided by P. Marchetti (Université de Lille II, France) (29). A375DDR double resistant variant was generated from A375 cells by repeated exposure to BRAF inhibitor Vemurafenib and MEK inhibitor Trametinib for 5/6 months until the onset of resistance. The murine melanoma primary cell lines Yumm1.7 (BrafV600E/Pten null/cdkn2a null) and MaNRas (NRasQ61K) were obtained from M. Bosenberg and L. Lame, respectively. Short-term patient melanoma cells MM099 and MM029 were kindly donated by J-C Marine and described elsewhere (30).

All cells were cultivated at 37° C. under 5% CO2 atmosphere. Human melanoma cells were grown in Dulbecco's modified Eagle's medium (DMEM) supplemented with 7% fetal bovine serum (FBS) (GE Healthcare HyClone), 50 U/ml penicillin and 50 μg/mL streptomycin. The Yumm1.7 cells were cultured in OPTIMEM medium (Thermo Fischer Scientific), 3% FBS and penicillin/streptomycin. The MaNRAS cells were maintained in HAM F12 medium supplemented with 10% FCS, penicillin/streptomycin and 100 nM PMA. All cell lines were routinely tested for the absence of mycoplasma by PCR.

Reagents and Antibodies

Culture reagents and DAPI were purchased from Thermo Fischer Scientific. Vemurafenib (PLX4032) and Trametinib (GSK1120212) were from Selleckchem. Chemicals, cycloheximide (CHX), 8TQ and puromycin were from Merck Millipore. The antibodies used for western blotting were as follow: PSMD14 (Generon), HSP90, p53 (Santa Cruz Biotechnology), K48-linkage specific polyubiquitin (D9D5), p21, CDK2, PARP, cleaved caspase 3, phospho-Thr202/Tyr204 Erk1/2 (Cell Signaling Technology), phospho-Ser139 H2AX (BioLegend). Alexa Fluor TexasRed-conjugated secondary antibodies were from ThermoFisher Scientific.

siRNA and Transfection

All siRNA were transfected using Lipofectamine RNAiMAX (ThermoFisher Scientific) following the manufacturer's recommendations. The siRNA screen was performed using a siGENOME® SMARTpool® siRNA Library—Human Deubiquitinating Enzymes (Catalog #G-104705-01) from Dharmacon (Horizon Discovery). 501Mel (1×105) were plated on 24-well plates. After 24h, cells were transfected with either 50 nM of non-targeting 50 nM of SMARTpool® siRNA or individual DUB-targeting SMARTpool® siRNA using Lipofectamine RNAiMAX. Cell proliferation was followed by live-cell imaging using the IncuCyte ZOOM™ system (Essen BioScience) for 72h. For the experimental validation of PSMD14 depletion, melanoma cells were transfected as described before (27) using 50 nM of Control siRNA (Universal negative controle #1 (SIC001) or 3 separate PSMD14 siRNA (SASI_Hs02_00340316, SASI_Hs01_00024446; SASI_Hs01_00024447) from Merck Millipore. The p53 siRNA was from Dharmacon (Horizon Discovery).

Immunofluorescence Staining

For phospho-H2AX (pH2AX) foci staining, cells grown on coverslips or culture dishes were fixed with 4% paraformaldehyde and permeabilized in 50 mM Tris-HCl, pH 7.4, 150 mM NaCl, 0.3% Triton X-100 for 1 h at 4° C. Cells were then blocked in 50 mM Tris-HCl, pH 7.4, 150 mM NaCl, 0.03% Triton X-100, 1% BSA for 1 h at 37° C., and stained with anti-phospho-Ser139 H2AX (1:1000, Merck Millipore #05-636) at 4° C. overnight. After extensive washings, cells were stained with Alexa Fluor TexasRed-conjugated secondary antibodies and 10 μg/ml DAPI for 1 h at room temperature. After washing, cells were mounted with ProLong Gold Antifade Mounting Oil (Thermo Fischer Scientific). The percentage of cells positive for phospho-H2AX foci was determined by analyzing 200 randomly chosen cells and the foci-positive cells were defined as nuclei with at least one large focus or a granular pattern of fluorescence.

Immunohistochemistry

Slices (4 μm) from paraffin-embedded tumors sections were Tissue sections were processed for dewaxing, rehydration and antigenic heat unmasking using the PT Module Lab Vision™ (ThermoFisher Scientific) according to the manufacturer's instructions. Tissue sections were then labelled with primary antibodies diluted in 1% BSA (wt/vol) followed by standard avidin-biotin-peroxidase staining (Vector Laboratories) using the automated staining system Myreva SS30 (Microm Microtech, France). Primary antibodies were as follows: phospho-H2AX (Merck Millipore), Ki67 (Agilent Dako), cleaved caspase 3 (Cell Signaling Technology).

Cell Proliferation Assays

After treatment, cells were fixed in PFA 3% during 20 min, washed with PBS and stained with crystal violet 0.4% in ethanol 20% for 30 min. After washing, crystal violet was solubilized from fixed cells. Its concentration was then estimated by optical density at 595 nm. Relative cell number was represented as the percentage of crystal violet concentration in control conditions. Alternatively, cell proliferation was followed by live-cell imaging using the IncuCyte ZOOM™ system (Essen BioScience) as described before (27). Phase contrast images were taken every hour over a 3-day period. Growth curves were generated using the IncuCyte™ cell proliferation assay software based on cell confluence. After 72h, cell proliferation in each condition was represented relative to control conditions.

Cell Viability Assays

After 24 to 48h of treatment, cell viability was evaluated by flow cytometry following staining with Annexin-V-FITC and propidium iodide (PI) (ThermoFisher Scientific) as previously described (27). The percentage of dead cells (Annexin-V-FITC+ and PI+ cells) was then determined. The same method was used for the determination of the half-maximal inhibitory concentration values (IC50) of 8-TQ on melanoma cells. Alternatively, cell viability was followed for 24h by live-cell imaging using the Red Cytotox fluorescent reagent and the IncuCyte ZOOM™ system (Essen BioScience) according to the manufacturer's instruction.

Animal Experimentation

All mouse experiments were carried out in accordance with the Institutional Animal Care and the local ethics committee (CIEPAL-Azur agreement NCE/2018-509). The animals were maintained on a 12h light/dark cycle in a temperature-controlled facility at 22° C. and were provided with free access to food (standard laboratory chow diet from UAR). For melanoma xenografts, 2×106 BRAF inhibitor resistant A375Riv human melanoma cells were subcutaneously implanted into both flanks of 6 week old athymic nude nu/nu female mice (Envigo). For melanoma syngeneic models, 2 to 4 105 of Yumm1.7 or MaNras murine melanoma cells were subcutaneously inoculated into 6 week old C57Bl/6J female mice (Envigo). When tumors became palpable (0.05 to 0.1 cm3), mice were injected intraperitoneally once every other day with vehicle or 10 mgkg-1 8TQ in in a 90:9:1 (v/v/v) mixture of Labrafil, dimethylacetamide, and Tween 80. The tumor size was measured with a caliper and the tumor volume was determined using the formula: Volume=(length×Width2)/2. At the end of the experiment, mice were sacrificed and tumors dissected, weighed, fixed and embedded in paraffin for IHC analysis.

Gene Expression Analysis

Data from The Cancer Genome Atlas (TCGA) (SKCM) were analyzed through the GEPIA web-based platform (http://gepia.cancer-pku.cn/) to analyze PSMD14 levels in normal and primary melanoma tissues. Additional data were extracted from Gene Expression Omnibus (GEO) database to examine PSMD14 expression across primary tumors and metastatic melanoma (GSE8401 and GSE46517) and during melanoma progression (GSE3189). Normalized data were analyzed using GraphPad Prism V5.0b software (GraphPad). For survival analyses, TCGA melanoma patient data were retrieved using the SurvExpress website (http://bioinformatica.mty.itesm.mx:8080/Biomatec/SurvivaX.jsp) (31). The SurvExpress database was used to compare the overall survival rates of individuals segregated according to PSMD14 expression. The risk groups generated from the SurvExpress database were based on the prognostic index (PI), and were split by the ordered PI (higher values for higher risk) with equal number of samples in each group. The PI was computed using the expression levels and values obtained from Cox fitting. Gene Set Enrichment Analysis (GSEA) of PSMD14high versus PSMD14low TCGA melanomas was performed using the GSEA software developed by the Broad Institute (http://software.broadinstitute.org/gsea/index.jsp) as described before (32).

Statistical Analysis

Unless otherwise stated experiments were repeated at least three times and representative data/images are shown. Statistical analysis was performed using the GraphPad Prism software. Data are presented as mean±SEM. For comparisons between two groups, P values were calculated using unpaired one-sided t-test or Mann-Whitney test. Statistical significance of the in vivo experiment was calculated with the two-way ANOVA test. P values of 0.05 (*), 0.01 (**) and 0.001 (***) were considered statistically significant.

Results

PSMD14 is a Novel Target and Prognostic Factor in Melanoma

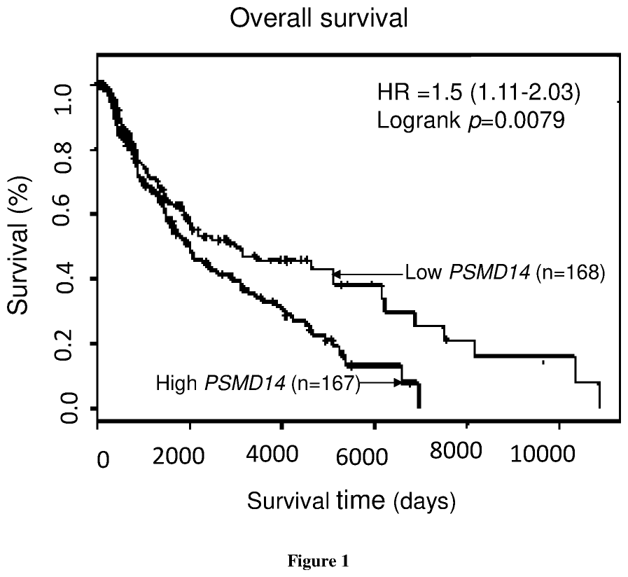

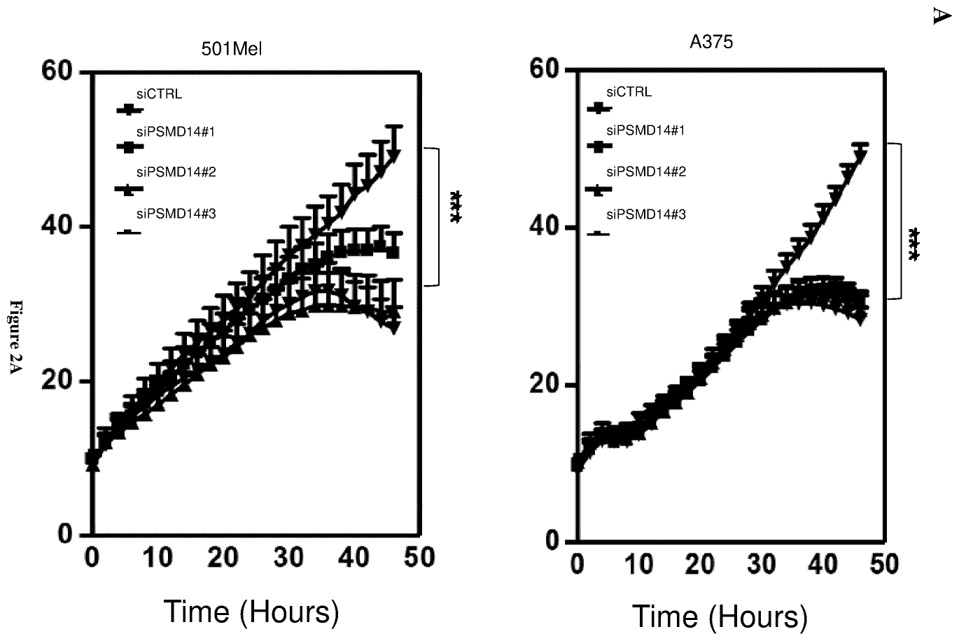

We undertook an unbiased genetic screen to identify DUB superfamily members that modulate melanoma cell proliferation and survival. We used 501Mel cells as a representative BRAF mutated melanoma cell line harbouring a proliferative phenotype (4). A human DUB siRNA library consisting of pools of four oligonucleotide sequences targeting 98 human DUBs (Dharmacon siGENOME® SMARTpool® siRNA Library—Human Deubiquitinating Enzymes) and non-targeting siRNAs were transfected into 501Mel cells and cell proliferation was followed for 96h by live-cell imaging of cell confluence (data not shown). We then assembled our results into a rank order based on the effect of each pools of siDUBs on cell proliferation at 72h compared to the mean of non-targeting control siRNA conditions (data not shown). Importantly, our screen confirmed the importance of USP14 for the regulation melanoma cell proliferation in agreement with our previous studies (27), thereby validating our approach. Nevertheless, the knockdown of 8 additional DUBs, including PSMD14, resulted in a more severe inhibition of 501Mel cell proliferation after 72h (>50% inhibition of cell proliferation). PSMD14 (also known as RPN11 and POH1) is a Zn2+-dependent metalloisopeptidase of the JAMM family that is an intrinsic component of the 19S regulatory particle of the proteasome (33,34). We therefore hypothesized that PSMD14 may constitute a key contributor of melanoma biology and therapeutic response. We analyzed the expression levels of PSMD14 in 451 cases of melanoma tissues and 558 cases of normal skin tissues using the Gene Expression Profiling Interactive Analysis (GEPIA) database (http://gepia.cancer-pku.cn/). The results indicated that PSMD14 mRNA was significantly upregulated in melanoma tissues compared to normal skin tissues (p<0.05, data not shown). Our in silico analysis of PSMD14 expression in public datasets (GSE8401 and GSE46517) further revealed an elevated expression of PSMD14 in malignant melanoma as compared to primary melanoma (data not shown). PSMD14 expression was also significantly correlated with melanoma progression as indicated by the higher expression detected in metastatic melanoma compared to benign skin lesions (nevi) (data not shown). Most importantly, the examination of PSMD14 gene expression in melanoma patients from The Cancer Genome Atlas (TCGA) datasets showed that high levels of PSMD14 were associated with a poorer overall survival (P=0.0079) (FIG. 1). Finally, GSEA analysis conducted on TCGA melanoma patient gene expression datasets indicated that PSMD14 levels are positively correlated with gene signatures of cell cycle checkpoints, cell proliferation and metabolic pathways, pathways that have been implicated in melanoma progression (data not shown). Together, these results identify PSMD14 as a novel potential target and prognostic factor in metastatic melanoma.

Expression of PSMD14 is Required for Melanoma Cell Proliferation and Survival

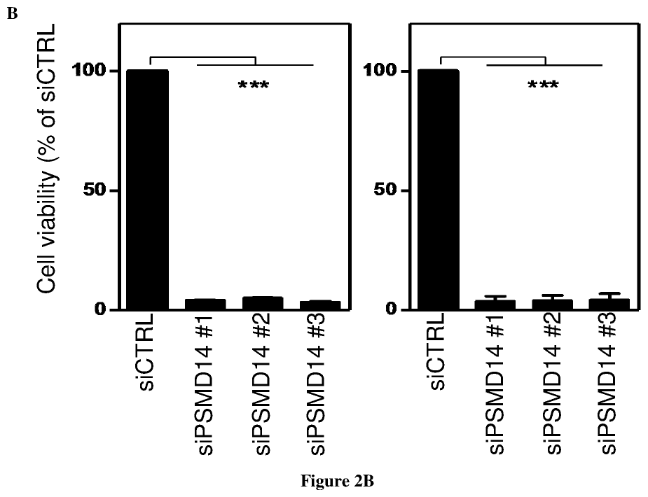

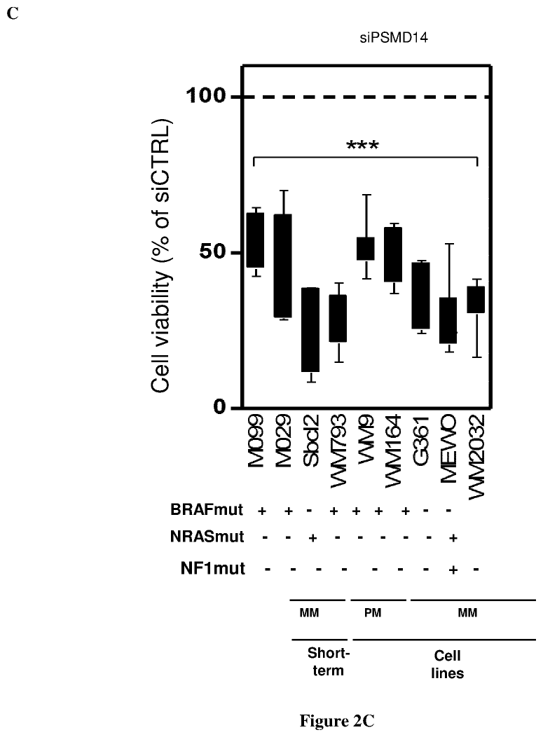

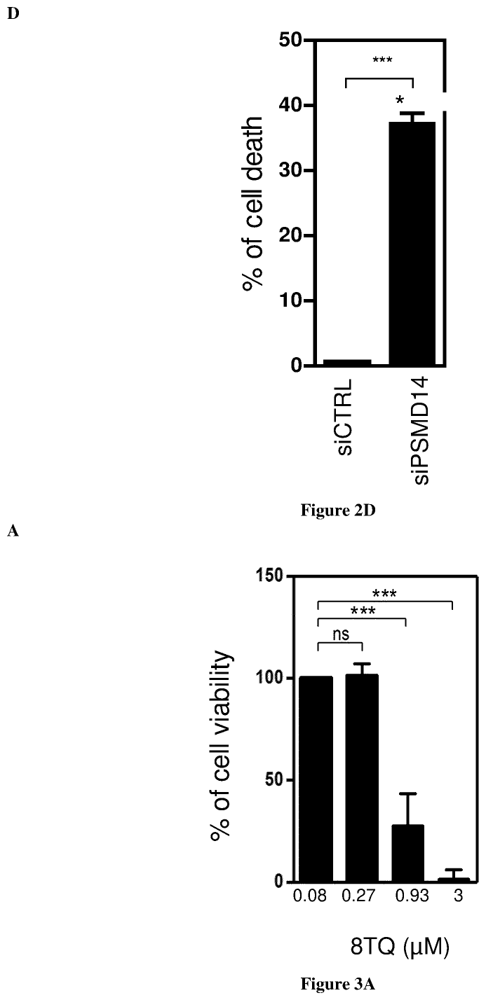

Previous studies have shown that PSMD14 confers a growth advantage in several types of cancer (34-37). To further investigate the role of PSMD14 in melanoma, we knocked down PSMD14 in BRAF mutant cell lines 501Mel and A375 by using three independent siRNA sequences and monitored cell proliferation by live-cell imaging. Consistent with the results of our screen, all three siPSMD14 sequences severely reduced melanoma cell proliferation after 30h to 48h of transfection (FIG. 2A). Growth inhibition induced by PSMD14 depletion was associated with the down-regulation of the cell-cycle dependent kinase CDK2 and a strong induction of the CDK inhibitor p21Cip1 (data not shown). A massive accumulation of polyubiquitylated proteins was also observed in PSMD14 depleted melanoma cells (data not shown), in agreement with previous studies that have attributed to PSMD14/RPN11 a major role in deubiquitination and proteasome-mediated protein degradation (38). Clonogenic growth is a hallmark of malignant cells. We therefore performed a colony formation assay with melanoma cell lines depleted or not of PSMD14. Compared with cells transfected with control siRNA, A375 and 501Mel cells transfected with PSMD14 siRNAs were no longer capable of forming colonies after 7 days, indicating that PSMD14 expression is required for melanoma clonogenic growth (FIG. 2B). We next examined the role of PSMD14 on cell survival of a collection of melanoma cell lines with diverse mutational status and metastatic potential and representative of three major subtypes of melanoma based on the frequency of BRAF, NRAS and NF1 mutations (3). PSMD14 was knocked-down in seven melanoma cell lines harbouring activating mutations in BRAF (WM793, WM9, G361) and in NRAS (Sbcl2, WM2032) or hemizygous deletion of NF1 (MEWO), as well as in two short term cultures from metastatic melanoma with BRAFV600E mutations (M029 and M099) (FIG. 2C). After 72h the crystal violet staining of cell cultures was quantified. Compared to siCTRL, we found that PSMD14 depletion strongly reduced melanoma cell viability regardless of the type of oncogenic driver mutation and regardless of their transcriptional cell state and their metastatic potential. Finally, flow cytometry analysis shows that the level of apoptotic cell death increased in A375 cells after 72h of PSMD14 depletion compared to control (FIG. 2D). Consistently, compared to siCTRL-transfected cells, siPSMD14-transfected A375 cells exhibited increased expression of the apoptosis-associated markers cleaved PARP and cleaved caspase-3) (data not shown).

Pharmacological Inhibition of PSMD14 Impairs Melanoma Cell Survival In Vitro and In Vivo

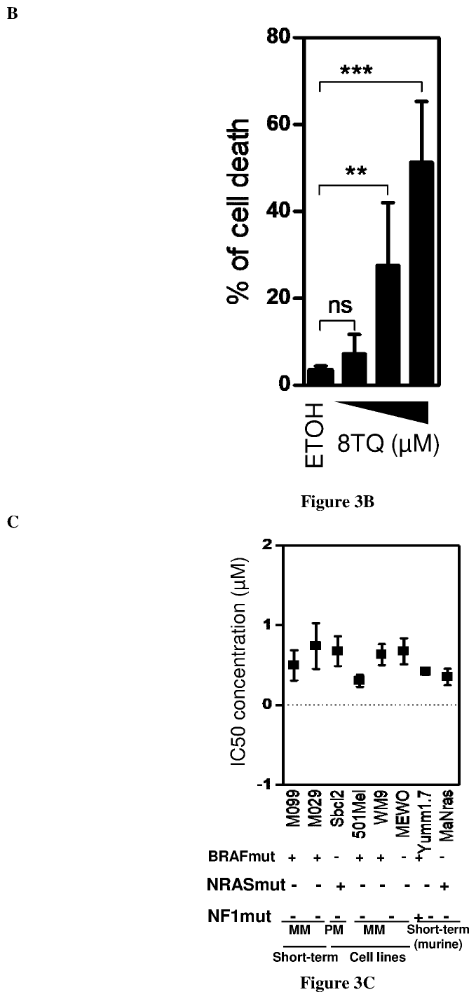

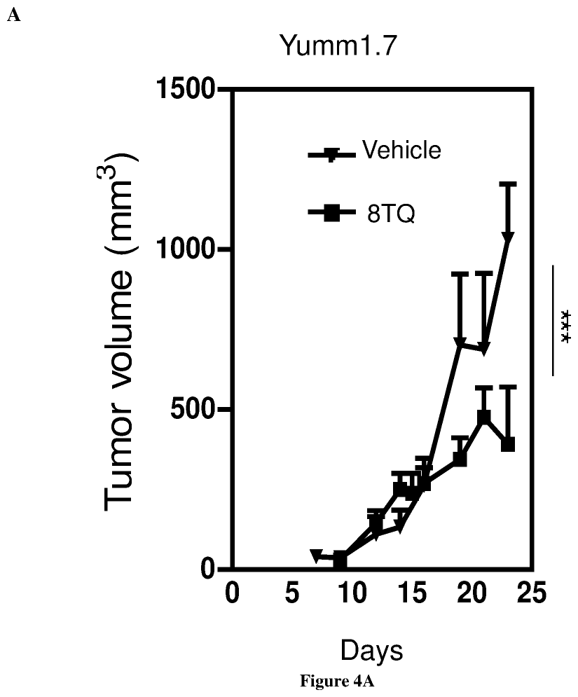

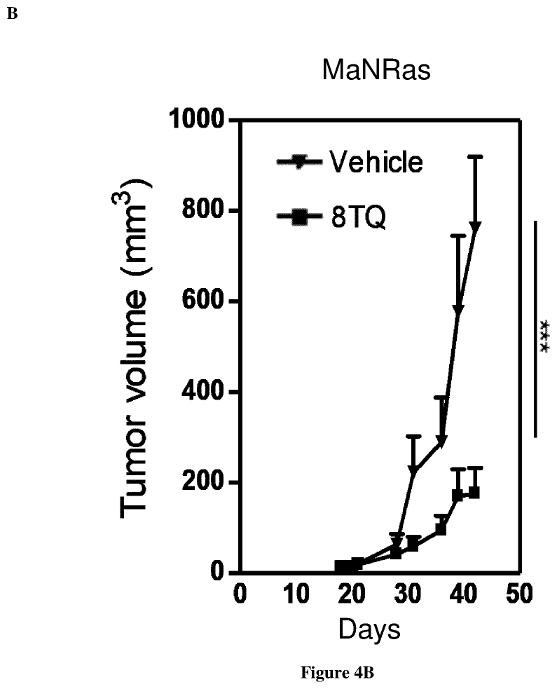

Keeping in mind a possible translation of our observations to the clinic, we investigated the action of PSMD14 on melanoma survival by using a selective inhibitor of the JAMM domain of PSMD14, 8-thioquinoline (8TQ) that was recently described (39). We treated A375 cells with increasing doses of 8TQ for 72h and found that pharmacological inhibition of PSMD14 by 8TQ had a pronounced cytotoxic effect on melanoma cell cultures, with a calculated IC50 for 8TQ of 0.66 μM (FIG. 3A). Consistently, the analysis by flow cytometry of Annexin V-FITC/DAPI staining performed on treated A375 cells revealed a dose-dependent increase of apoptotic cell death (FIG. 3B). Using the same approach, we calculated the IC50 of 8TQ on a collection of human (M099, M029, Sbcl2, 501Mel, WM9 and MEWO) and murine (Yumm1.7 and MaNras) melanoma cell lines and short-term cultures to be in a μM range (0.25 to 0.75 μM) (FIG. 3C). Importantly, 8TQ markedly inhibited melanoma cell viability irrespective of the mutational status or the disease stage. Western blot analysis next showed that treatment with 8TQ induced the accumulation of poly-ubiquitinated proteins (data not shown) and the expression of cleaved PARP and cleaved caspase-3 (data not shown) in A375 cells, supporting the notion that inhibition of PSMD14 decreases melanoma cell viability through a proteotoxic stress-mediated apoptotic process. To investigate whether PSMD14 regulates melanoma growth in vivo, we tested the effect of 8TQ treatment on two syngeneic murine melanoma models. We employed the Yumm1.7 cells and MaNRas cells, which derive from BrafV600E/Pten null/cdkn2a null mice (40) and NRasQ61K mice (L. Lame, Institut Curie, France, unpublished data), respectively. Importantly, both Yumm1.7 and MaNRAS cells showed similar sensitivity to PSMD14 inhibition compared to human melanoma cells (FIG. 3C). Yumm1.7 cells and MaNRas cells were subcutaneously transplanted into immunocompetent C57Bl/6J mice. Injected mice were then treated with 8TQ or vehicule and tumor growth was monitored. We found that compared to vehicule, 8TQ significantly decreased both Yumm1.7 (FIG. 4A) and MaNRAS tumor growth (FIG. 4B). Consistently, the analysis of Yumm1.7 tumors dissected from 8TQ-treated mice at the experiment endpoint showed that both tumor size and weight were markedly reduced compared to vehicule-treated animals (data not shown). Immunostaining on tumor sections further showed that the reduction of tumor growth upon 8TQ treatment was accompanied by a decreased staining of the cell proliferation marker Ki67, and conversely by an increased staining of the apoptosis marker cleaved caspase 3 (data not shown). Altogether, these results demonstrate that PSMD14 plays a critical role in melanoma cell survival in vitro and in vivo.

Targeting PSMD14 Overcomes Resistance to MAPK Pathway Inhibitors

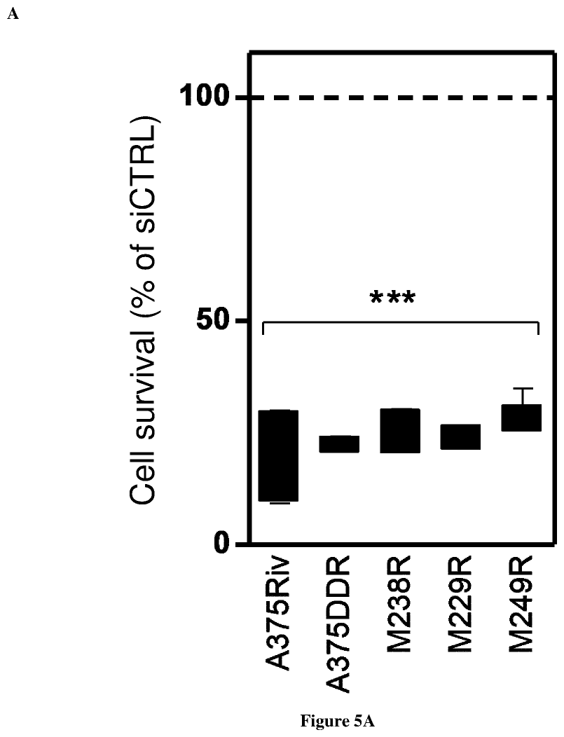

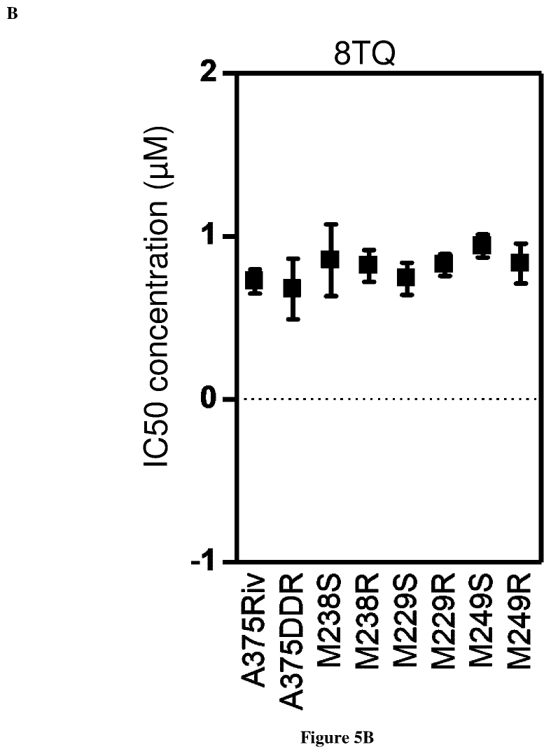

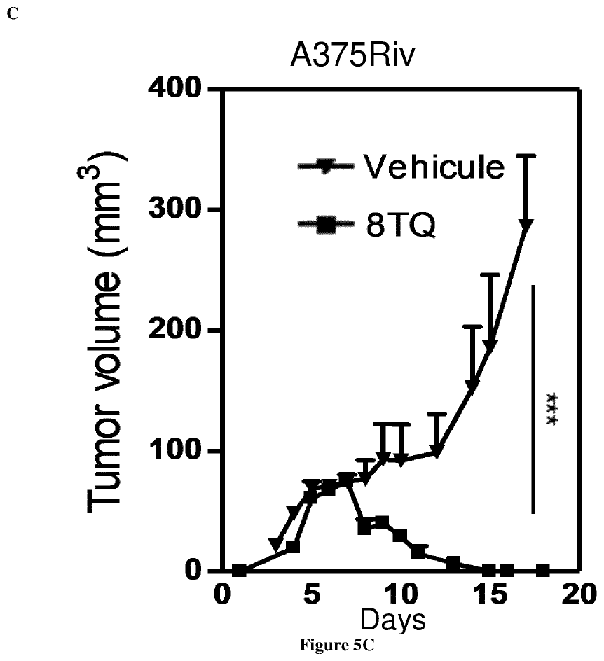

Resistance to BRAF/MEK inhibitors remains a major challenge for long-term clinical benefit of targeted therapies in advanced metastatic BRAF mutated melanomas (9,10). Therefore we tested whether PSMD14 inhibition could represent a therapeutically exploitable option against minimal residual disease (MRD) and acquired resistance that are induced by targeted therapies (5,41). To this end, we developed an in vitro model of therapeutic resistance during which A375 cells were treated with different doses of the combination of dabrafenib and trametinib for up to 6 months. Following the early therapeutic response (1 to 7 days), a phase similar to MRD (2 to 3 weeks) contributes to the persistence of drug tolerant cells (DTC), some of which will eventually acquire drug resistance through adaptive molecular processes after 4 to 6 months (data not shown). Western blot analysis of cells collected during representative phases of the process of drug resistance acquisition showed that PSMD14 expression decreased upon short exposure to BRAFi/MEKi combination (data not shown). As expected, BRAFi/MEKi treatment rapidly decreased the phosphorylation of the BRAF/MEK substrate ERK1/2. Interestingly, in parallel to ERK1/2 phosphorylation, PSMD14 expression was restored in DTC and in A375DDR resistant cells (data not shown). This observation led us to hypothesize that DTC exposure to 8TQ might prevent late drug resistance. A375 cells were treated with cytotoxic doses of dabrafenib and trametinib for 14 days. 1 μM 8TQ was then added or not to the cell culture for an additional 7 days and plates were stained with crystal violet to examine the persistence of DTC. In the absence of co-treatment with 8TQ, a significant number of drug tolerant colonies were observed upon 21 days of dabrafenib/trametinib combination (7 nM and 24 nM). In contrast, no DTC were observed upon dabrafenib/trametinib/8TQ combination (data not shown), suggesting that PSMD14 is required for MRD and acquired resistance in targeted melanoma therapy. Consistent with this notion, we found that siRNA-mediated depletion of PSMD14 impaired the viability of BRAFi-resistant cell lines (M229R, M238R, M249R and A375Riv) (11) (29), and of the double resistant A375 subline that we generated (A375DDR) (data not shown and 5A). Furthermore, PSMD14 inhibition with doses of 8TQ potently decreased the viability of the same collection of BRAFi- and BRAFi/MEKi-resistant cells. The IC50 of 8TQ calculated from these experiments was in the same μM range than their parental counterparts (FIG. 5B). Finally, we examined the impact of PSMD14 inhibition on the growth of BRAFi-resistant xenografts. A375Riv cells were subcutaneously implanted into nude mice and tumor growth in animals treated or not with 8TQ was monitored. Compared to vehicule, PSMD14 inhibitor dramatically abrogated tumor development (FIG. 5C). Together, these results indicate that targeting PSMD14 prevents the development of drug tolerance and overcomes acquired resistance to MAPK signalling inhibitors.

PSMD14 Inhibition Triggers a p53-Dependent DNA Damage Response in Melanoma

In silico analysis of melanoma datasets correlated high levels of PSMD14 to gene signatures of cell cycle checkpoints and E2F targets (data not shown). E2F1 is an important regulator of DNA damage response and cell survival in several types of cancer including melanoma (42). In addition, it has been shown that PSMD14 is part of a proteosomal complex colocalizing with DNA damage foci (43). We therefore examined whether cell death in response to PSMD14 inhibition involves an unchecked DNA damage response in melanoma. The upregulation of p53 and the phosphorylation of histone H2AX (pH2AX) are among the earliest responses to DNA double strand breaks (DSBs). PSMD14 knockdown increased both p53 expression and H2AX phosphorylation in a time dependent manner, indicating that PSMD14 expression is required for the resolution of DNA damage in melanoma. Consistently, immunofluorescence analysis revealed that H2AX phosphorylated at serine 139 localized into the nucleus and formed punctate foci upon PSMD14 depletion (Data not shown and 6A), reflecting the presence of local DNA DSBs in the absence of PSMD14.

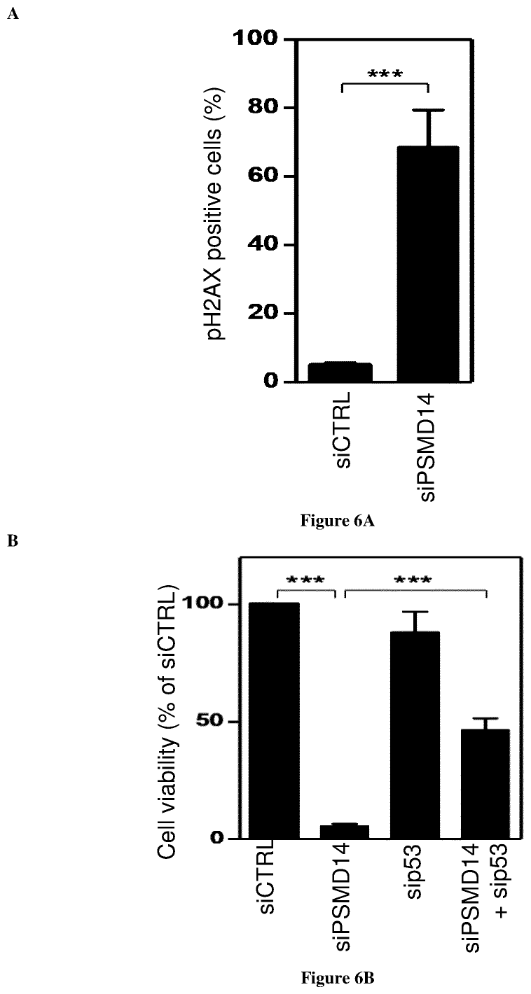

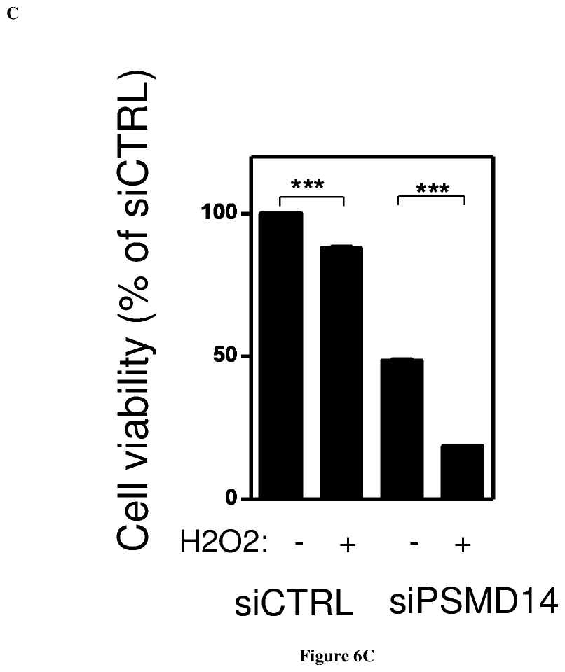

In addition, we observed that 501Mel and A375 melanoma cells that were exposed to increased doses of 8TQ displayed an elevated expression of phosphorylated H2AX after 48h associated to visible punctuated foci par immunofluorescence (data not shown). The tumor suppressor p53 is a major effector of DNA damage-associated signaling pathways. Because targeting PSMD14 induced a strong increase of p53, we examined its involvement in cell death caused by PSMD14 depletion. A375 cells were transfected for 72h with siCTRL, siPSMD14, sip53 or the combination of siPSMD14 and sip53 and cell survival was evaluated by quantification of crystal violet staining. We observed that the knock-down of p53 significantly rescued PSMD14-depleted melanoma cells from cell death (FIG. 6B). To get further insights into the role of PSMD14 in DNA damage response in melanoma, we stimulated control or PSMD14-depleted cells with H2O2, an oxidative stress inducer that is known to induce DNA damage. Interestingly, PSMD14 knockdown in A375 cells cooperated with H2O2 treatment to further increase oxidative stress-induced cell death (FIG. 6C). Taken together, these results indicate that the deubiquitinating activity of PSMD14 is required for maintaining genome integrity and cell viability.

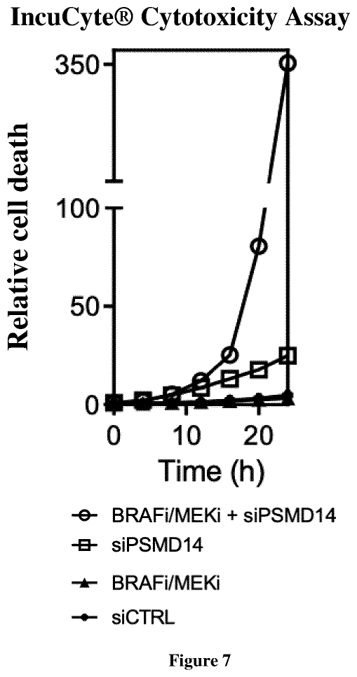

Targeting PSMD14 has a synergistic action with the combination of BRAFi and MEKi on BRAF mutant melanoma cell viability. Finally, we tested whether targeting PSMD14 could potentiate the therapeutic action of the combination of BRAF and MEK inhibitors, which represents the frontline treatment for patients with BRAF mutant melanomas (9, 10). BRAF mutant A375 cells were transfected with control siRNA (siCTRL) or siRNA targeting PSMD14, and incubated in the presence of DMSO or a combination of Vemurafenib (BRAFi) and Cobimetinib (MEKi). Cell death was then quantified during 24h using a fluorescent assay and live cell imaging. As shown in FIG. 7, whereas the combination BRAFi/MEKi or short term PSMD14 knockdown alone (siPSMD14) had only marginal effect on cell viability, exposure to BRAFi and MEKi dramatically triggered cell death on PSMD14 depleted melanoma cells (BRAFi/MEKi+siPSMD14). These data indicate that targeting PSMD14 synergizes the cytotoxic action of BRAFi/MEKi combination on BRAF mutant melanoma cell in vitro, and suggest that small molecule inhibitors of PSMD14 may improve the current targeted therapies.

### REFERENCES

Throughout this application, various references describe the state of the art to which this invention pertains. The disclosures of these references are hereby incorporated by reference into the present disclosure.
1. Miller A J, Mihm M C, Jr. Melanoma. N Engl J Med 2006; 355:51-65
2. Shain A H, Bastian B C. From melanocytes to melanomas. Nat Rev Cancer 2016; 16:345-58
3. Cancer Genome Atlas N. Genomic Classification of Cutaneous Melanoma. Cell 2015; 161:1681-96
4. Widmer D S, Cheng P F, Eichhoff O M, Belloni B C, Zipser M C, Schlegel N C, et al. Systematic classification of melanoma cells by phenotype-specific gene expression mapping. Pigment Cell Melanoma Res 2012; 25:343-53
5. Tsoi J, Robert L, Paraiso K, Galvan C, Sheu K M, Lay J, et al. Multi-stage Differentiation Defines Melanoma Subtypes with Differential Vulnerability to Drug-Induced Iron-Dependent Oxidative Stress. Cancer Cell 2018; 33:890-904 e5
6. Shaffer S M, Dunagin M C, Torborg S R, Torre E A, Emert B, Krepler C, et al. Rare cell variability and drug-induced reprogramming as a mode of cancer drug resistance. Nature 2017; 546:431-5
7. Straussman R, Morikawa T, Shee K, Barzily-Rokni M, Qian Z R, Du J, et al. Tumour micro-environment elicits innate resistance to RAF inhibitors through HGF secretion. Nature 2012; 487:500-4
8. Young H L, Rowling E J, Bugatti M, Giurisato E, Luheshi N, Arozarena I, et al. An adaptive signaling network in melanoma inflammatory niches confers tolerance to MAPK signaling inhibition. J Exp Med 2017; 214:1691-710
9. Flaherty K T, Hodi F S, Fisher D E. From genes to drugs: targeted strategies for melanoma. Nat Rev Cancer 2012; 12:349-61
10. Long G V, Weber J S, Infante J R, Kim K B, Daud A, Gonzalez R, et al. Overall Survival and Durable Responses in Patients With BRAF V600-Mutant Metastatic Melanoma Receiving Dabrafenib Combined With Trametinib. J Clin Oncol 2016; 34:871-8
11. Nazarian R, Shi H, Wang Q, Kong X, Koya R C, Lee H, et al. Melanomas acquire resistance to B-RAF(V600E) inhibition by RTK or N-RAS upregulation. Nature 2010; 468:973-7
12. Kemper K, Krijgsman O, Cornelissen-Steijger P, Shahrabi A, Weeber F, Song J Y, et al. Intra- and inter-tumor heterogeneity in a vemurafenib-resistant melanoma patient and derived xenografts. EMBO Mol Med 2015; 7:1104-18
13. Smith M P, Sanchez-Laorden B, O'Brien K, Brunton H, Ferguson J, Young H, et al. The immune microenvironment confers resistance to MAPK pathway inhibitors through macrophage-derived TNFalpha. Cancer Discov 2014; 4:1214-29
14. Hirata E, Girotti M R, Viros A, Hooper S, Spencer-Dene B, Matsuda M, et al. Intravital imaging reveals how BRAF inhibition generates drug-tolerant microenvironments with high integrin beta1/FAK signaling. Cancer Cell 2015; 27:574-88
15. Eggermont A M, Spatz A, Robert C. Cutaneous melanoma. Lancet 2014; 383:816-27
16. Balch W E, Morimoto R I, Dillin A, Kelly J W. Adapting proteostasis for disease intervention. Science 2008; 319:916-9
17. Finley D. Recognition and processing of ubiquitin-protein conjugates by the proteasome. Annu Rev Biochem 2009; 78:477-513
18. Nijman S M, Luna-Vargas M P, Velds A, Brummelkamp T R, Dirac A M, Sixma T K, et al. A genomic and functional inventory of deubiquitinating enzymes. Cell 2005; 123:773-86
19. Reyes-Turcu F E, Ventii K H, Wilkinson K D. Regulation and cellular roles of ubiquitin-specific deubiquitinating enzymes. Annu Rev Biochem 2009; 78:363-97
20. Kumari N, Jaynes P W, Saei A, Iyengar P V, Richard J L C, Eichhorn P J A. The roles of ubiquitin modifying enzymes in neoplastic disease. Biochim Biophys Acta Rev Cancer 2017; 1868:456-83
21. Ma J, Guo W, Li C. Ubiquitination in melanoma pathogenesis and treatment. Cancer Med 2017; 6:1362-77
22. Zhao X, Fiske B, Kawakami A, Li J, Fisher D E. Regulation of MITF stability by the USP13 deubiquitinase. Nat Commun 2011; 2:414
23. Potu H, Peterson L F, Kandarpa M, Pal A, Sun H, Durham A, et al. Usp9x regulates Ets-1 ubiquitination and stability to control NRAS expression and tumorigenicity in melanoma. Nat Commun 2017; 8:14449
24. Potu H, Peterson L F, Pal A, Verhaegen M, Cao J, Talpaz M, et al. Usp5 links suppression of p53 and FAS levels in melanoma to the BRAF pathway. Oncotarget
25. Jeong M, Lee E W, Seong D, Seo J, Kim J H, Grootjans S, et al. USP8 suppresses death receptor-mediated apoptosis by enhancing FLIPL stability. Oncogene 2017; 36:458-70
26. Zou Q, Jin J, Hu H, Li H S, Romano S, Xiao Y, et al. USP15 stabilizes MDM2 to mediate cancer-cell survival and inhibit antitumor T cell responses. Nat Immunol 2014; 15:562-70
27. Didier R, Mallavialle A, Ben Jouira R, Domdom M A, Tichet M, Auberger P, et al. Targeting the Proteasome-Associated Deubiquitinating Enzyme USP14 Impairs Melanoma Cell Survival and Overcomes Resistance to MAPK-Targeting Therapies. Mol Cancer Ther 2018; 17:1416-29
28. Massoumi R, Kuphal S, Hellerbrand C, Haas B, Wild P, Spruss T, et al. Down-regulation of CYLD expression by Snail promotes tumor progression in malignant melanoma. J Exp Med 2009; 206:221-32
29. Corazao-Rozas P, Guerreschi P, Jendoubi M, Andre F, Jonneaux A, Scalbert C, et al. Mitochondrial oxidative stress is the Achille's heel of melanoma cells resistant to Braf-mutant inhibitor. Oncotarget 2013; 4:1986-98
30. Verfaillie A, Imrichova H, Atak Z K, Dewaele M, Rambow F, Hulselmans G, et al. Decoding the regulatory landscape of melanoma reveals TEADS as regulators of the invasive cell state. Nat Commun 2015; 6:6683
31. Aguirre-Gamboa R, Gomez-Rueda H, Martinez-Ledesma E, Martinez-Torteya A, Chacolla-Huaringa R, Rodriguez-Barrientos A, et al. SurvExpress: an online biomarker validation tool and database for cancer gene expression data using survival analysis. PLoS One 2013; 8:e74250
32. Ohanna M, Cerezo M, Nottet N, Bille K, Didier R, Beranger G, et al. Pivotal role of NAMPT in the switch of melanoma cells toward an invasive and drug-resistant phenotype. Genes Dev 2018; 32:448-61
33. Spataro V, Simmen K, Realini C A. The essential 26S proteasome subunit Rpn11 confers multidrug resistance to mammalian cells. Anticancer Res 2002; 22:3905-9
34. Gallery M, Blank J L, Lin Y, Gutierrez J A, Pulido J C, Rappoli D, et al. The JAMM motif of human deubiquitinase Pohl is essential for cell viability. Mol Cancer Ther 2007; 6:262-8
35. D'Arcy P, Wang X, Linder S. Deubiquitinase inhibition as a cancer therapeutic strategy. Pharmacol Ther 2015; 147:32-54
36. Chen W, Guo N, Qi M, Dai H, Hong M, Guan L, et al. Discovery, mechanism and metabolism studies of 2,3-difluorophenyl-linker-containing PARP1 inhibitors with enhanced in vivo efficacy for cancer therapy. Eur J Med Chem 2017; 138:514-31
37. Luo G, Hu N, Xia X, Zhou J, Ye C. RPN11 deubiquitinase promotes proliferation and migration of breast cancer cells. Mol Med Rep 2017; 16:331-8
38. Verma R, Aravind L, Oania R, McDonald W H, Yates J R, 3rd, Koonin E V, et al. Role of Rpn11 metalloprotease in deubiquitination and degradation by the 26S proteasome. Science 2002; 298:611-5
39. Perez C, Li J, Parlati F, Rouffet M, Ma Y, Mackinnon A L, et al. Discovery of an Inhibitor of the Proteasome Subunit Rpn11. J Med Chem 2017; 60:1343-61
40. Meeth K, Wang J X, Micevic G, Damsky W, Bosenberg M W. The YUMM lines: a series of congenic mouse melanoma cell lines with defined genetic alterations. Pigment Cell Melanoma Res 2016; 29:590-7
41. Rambow F, Rogiers A, Marin-Bejar O, Aibar S, Femel J, Dewaele M, et al. Toward Minimal Residual Disease-Directed Therapy in Melanoma. Cell 2018; 174:843-55 e19
42. Rouaud F, Hamouda-Tekaya N, Cerezo M, Abbe P, Zangari J, Hofman V, et al. E2F1 inhibition mediates cell death of metastatic melanoma. Cell Death Dis 2018; 9:527
43. Tsolou A, Nelson G, Trachana V, Chondrogianni N, Saretzki G, von Zglinicki T, et al. The 19S proteasome subunit Rpn7 stabilizes DNA damage foci upon genotoxic insult. IUBMB Life 2012; 64:432-42

1. (canceled)

2. A method for treating melanoma in a subject in need thereof comprising administering to said subject a therapeutically effective amount of an inhibitor of PSMD14.

3. The method according to claim 2, wherein, the subject is identified as having a short survival time i) quantifying the expression level of PSMD14 in a biological sample obtained from the subject; ii) comparing the expression level quantified at step i) with its predetermined reference value and iii) concluding that the subject will have a short survival time when the expression level of PSMD14 is higher than its predetermined reference value.

4. The method according to claim 2, wherein, the inhibitor of PSMD14 is a small organic molecule.

5. The method according to claim 4 wherein, the small molecule is quinoline-8-thiol (8TQ).

6. The method according to claim 2 wherein, the inhibitor of PSMD14 is siRNA.

7. A method for treating melanoma in a subject in need thereof comprising administering to said subject a therapeutically effective amount of an inhibitor of PSMD14, an inhibitor of BRAF and an inhibitor of MEK.

8. A method for treating resistant melanoma in a subject in need thereof comprising administering to said subject a therapeutically effective amount of an inhibitor of PSMD14.

9. The method according to claim 8, wherein, the melanoma is resistant to a treatment with the inhibitors of BRAF mutations.

10. The method according to claim 8, wherein, the melanoma is resistant to a treatment with inhibitors of MEK.

11. The method according to claim 8, wherein, the melanoma is resistant to a treatment with inhibitors of NRAS.

12. The method according to claim 8, wherein, the melanoma is a resistant double-negative BRAF and NRAS mutant melanoma.

13. The method according to claim 8, wherein, the melanoma is resistant to a treatment with an immune checkpoint inhibitor.

14. The method of claim 8, further comprising administering to said subject an inhibitor of BRAF and an inhibitor of MEK.

15. (canceled)
